(19) United States
(12) Patent Application Publication
(10) Pub. No.: US 2010/0297618 A1
(43) Pub. Date: Nov. 25, 2010

(54) METHODS FOR DETERMINING A PROGNOSIS OF COLORECTAL CANCER

(76) Inventors: Christopher Hutchison, Durham (GB); Thomas Cox, Durham (GB); Robert Wilson, Yorkshire (GB); Stefan Przyborski, Durham (GB); Manon Van Engeland, Maastricht (NL); Adrian De Bruine, Maastricht (NL)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/305,682

(22) PCT Filed: Jun. 21, 2007
(86) PCT No.: PCT/GB2007/002316

§ 371 Date: Jul. 15, 2010

Related U.S. Application Data
(60) Provisional application No. 60/815,921, filed on Jun. 23, 2006.

(51) Int. Cl. C12Q 1/68; G01N 33/53; C12Q 1/02

(52) U.S. Cl. 435/6; 435/7.1; 435/29

(57) ABSTRACT

The invention provides methods for determining a prognosis of colorectal cancer in an individual comprising determining an A-type lamin status of an individual and using the A-type lamin status to determine the prognosis of the colorectal cancer, and more particularly for predicting outcomes of colorectal cancer in the individual such as the survival or mortality of the individual or the likelihood of recurrence of the colorectal cancer. Kits for use in carrying out these methods are also provided.

### FIELD OF THE INVENTION

The present invention relates to methods for determining a prognosis of colorectal cancer in an individual using a novel prognostic marker, and more particularly to predicting outcomes of colorectal cancer in the individual such as the survival or mortality of the individual or the likelihood of recurrence of the colorectal cancer. These findings may be used to help to determine appropriate treatments for patients with colorectal cancer.

### BACKGROUND OF THE INVENTION

Colorectal cancer is a highly prevalent form of cancer for which both improved treatments and clinical markers to predict disease outcome are desired. Recent estimates indicate that there are around 165,000 new cases of colorectal cancer per year in the United States and 370,000 in Europe. Colorectal cancer is therefore a major public health issue and new technologies that can improve the clinical management of this disease would be highly desirable.

As with many cancers, the early detection of colorectal cancer increases the likelihood that the tumour can be successfully removed and the cancer cured. After diagnosis, colorectal tumours are normally treated surgically and this is often curative if the tumour is removed prior to the development of a more aggressive phenotype. Colorectal cancers are graded clinically and this information helps clinicians to plan the most appropriate treatment strategy for each individual patient.

There are two major grading systems in use for colorectal cancer. Colorectal cancers are normally graded according to the Dukes scale. This scale contains four grades; A, B, C and D. Dukes C and Dukes D cases are the most advanced stages and such patients generally have a poor long term outlook despite the widespread use of treatments such as adjuvant chemotherapy in addition to surgical intervention. In contrast, patients with Dukes grade A colorectal cancer are nearly always cured by surgical intervention alone. The majority (around 70%) of Dukes Grade B cases are also cured by surgical resection of the tumour, but around 30% of grade B cases suffer recurrences of the colorectal cancer leading to the death of the patient.

The alternative colorectal cancer grading system which is becoming commonly used is the TNM grading system. This system is based on the primary tumour size (T), extent of spread to lymph nodes (N) and the presence of metastasis (M), and together these criteria are used to define the clinical stage of the colorectal cancer. The TNM stage can be correlated with the Dukes staging system and those TNM stage I patients with tumour extension into the muscularis propria as well as the TNM stage II patients are generally categorised as Dukes grade B (Sobin 2002).

A wide range of mutations have been described which are associated with colorectal cancer (Bienz 2000). In contrast to this, there is a distinct lack of markers that can be used to accurately predict which colorectal cancer patients will ultimately suffer a recurrence and die from the disease and which will remain disease free in the long term. Such information would be extremely useful clinically as it would allow the identification of high-risk individuals who could be given more aggressive treatment regimes and monitored on a more regular basis. Although such a prognostic marker would be useful across all grades of colorectal cancer, it would be of particular value in the Dukes grade B cohort of patients.

There has been widespread discussion as to the merits of treating Dukes grade B patients with adjuvant chemotherapy alongside surgical intervention. Clinical studies have shown a small survival benefit of providing adjuvant chemotherapy to Dukes grade B patients (Gray 2004). As the observed survival benefit was relatively small there has been a reluctance to prescribe adjuvant chemotherapy as a standard treatment strategy for all Dukes grade B patients. The sub-population of Dukes grade B patients who suffer recurrence of their colorectal cancer and subsequent mortality may in fact derive greater clinical benefit from adjuvant chemotherapy than that observed when the population of grade B patients is considered as a whole. Markers that can prospectively identify this patient sub-population at the point of diagnosis/initial treatment would be extremely useful and could enable the use of adjuvant chemotherapy as a standard treatment for this high-risk group of patients.

The Lamin A/C gene encodes a number of intermediate type filament proteins, the A-type lamins, which are major components of the nuclear lamina (Hozak 1995). The nuclear lamina is a meshwork of filaments found on the nucleoplasmic side of the nuclear membrane. Nuclear A-type lamins have a range of roles including structural and regulatory functions. The Lamin A/C gene contains 12 exons and alternative splicing within exon 10 of the lamin A/C messenger RNA gives rise to two proteins; lamin A and lamin C (Lin 1993). The human lamin A protein is 664 amino acids in length whereas the lamin C protein contains 572 amino acids. The first 566 amino acid residues are common to both lamin A and C. In addition to lamin A and lamin C there is a third splice variant which is identical to lamin A, but which lacks the exon 10 sequence. This form gives rise to a 634 amino acid protein called lamin AΔ10 (Machiels 1996).

Changes in expression of the Lamin A/C gene have been reported in a number of cancers and A-type lamin expression has been reported to be downregulated in small cell lung carcinomas (Broers and Ramaekers 1994) and in testicular cancer (Machiels 1997). Loss of A-type lamins at the protein level has been associated with a highly proliferative status in basal cell carcinomas (Venables 2001). In primary colon carcinomas and adenomas, it has been observed that the expression of Lamin A/C is reduced and frequently undetectable by immunohistochemistry and in a number of cancers mislocalisation of A-type lamins to the cytoplasm of the cells has been detected (Moss 1999). Further studies have shown that the silencing of Lamin A/C gene expression by promoter hypermethylation occurs in Nodal diffuse large B-cell lymphoma and that this is associated with a decrease in patient survival (Agrelo 2005). From the literature, loss or mislocalisation of A-type lamins therefore appears to have negative connotations with respect to cancer due to the association of loss/mislocalisation of A-type lamins with more highly proliferative cellular behaviour and decreased patient survival.

### SUMMARY OF THE INVENTION

Broadly, the present invention is based on experiments that demonstrate that the expression and cellular localisation of the A-type lamin proteins from the Lamin A/C gene is indicative of the prognosis of colorectal cancer patients. More particularly, the work described herein confirms that the loss or mislocalisation of A-type lamin proteins correlates with a positive prognosis (long term survival and reduced chance of disease recurrence), whereas the presence of A-type lamin proteins in their normal nuclear localisation pattern is indicative of a poor prognosis (recurrence of disease and colorectal cancer associated mortality) for colorectal cancer patients. Accordingly, the determination of A-type lamin status, for example by determining the expression of A-type lamin proteins, Lamin A/C gene expression and/or analysis of the A-type lamin protein cellular localisation in tumour samples may therefore be used as a prognostic indicator of colorectal cancer patient outcome and thus help to determine the most appropriate clinical treatment strategy for the patient.

Thus, the present invention provides a means to define the clinical prognosis of colorectal cancer patients. The results may be contrasted with the findings reported in the literature that suggest that an absence of Lamin A/C is likely to be a poor prognostic indicator. The work described herein shows that the opposite is in fact the case and that the presence of Lamin A/C is an indicator of poor prognosis in colorectal cancer patients.

Accordingly, the present invention may be used to help in the prediction of colorectal cancer related mortality in colorectal cancer patients. In this study, colorectal cancer related mortality equates to all patients who death could be directly attributed to colorectal cancer as opposed to those who either survived or died due to an unrelated cause. In most instances, the prognostic prediction of colorectal cancer related mortality is equivalent to prediction of colorectal cancer recurrence and this invention can therefore be used to identify a high risk group of patients with an increased likelihood of both recurrence of colorectal cancer and ultimately colorectal cancer related mortality.

Accordingly, in a first aspect, the present invention provides a method for determining a prognosis of colorectal cancer in an individual, the method comprising determining an A-type lamin status of the individual and using the A-type lamin status to determine the prognosis of the colorectal cancer.

Preferably, the A-type lamin status of the individual is used for predicting the outcome of the colorectal cancer in that individual. By way of example, the outcome of the colorectal cancer may be predicting colorectal cancer associated survival or mortality of the individual and/or predicting the probability of long term survival of the individual and/or predicting recurrence of the colorectal cancer. Alternatively or additionally, the A-type lamin status may be used for determining the clinical treatment of the individual. By way of example, this may be of help in selecting a type of chemotherapy or a chemotherapy regimen for administration to the individual and/or in determining whether a colorectal tumour should be treated by surgical resection.

As set out above, the assignment of individuals to the existing Dukes scale and TNM grading system for colorectal cancer often fails to adequately distinguish some of the patients who will respond well to treatment and remain disease free and other groups of patients for whom a more aggressive treatment regimen is needed. The methods disclosed herein may be used to help to solve these problems, and so provide appropriate treatment to individuals with colorectal cancer. This may be by using the results of the method of prognosis of the present invention alone to determine appropriate treatment for a patient or by combining the results of the prognostic method of the present invention with an existing grading systems for patients with colorectal cancer. In particular, the present method may be used to help to improve the prediction of outcome of patients categorised as grade B on the Dukes scale or the equivalent TNM stage II patients on the TNM grading system. A-type lamin status may also be employed for predicting recurrence of early grade or Dukes stage A colorectal cancer or adenomas.

Preferably, determining the A-type lamin status of the individual comprises determining the expression of the Lamin A/C gene, and/or the presence, amount and/or localisation of an A-type lamin protein, or a fragment thereof. Both determinations may include assessing the prevalence of the A-type lamin status in question in the tumour cells, that is, how many of the cells in a sample under consideration share the A-type lamin status in question.

As explained in more detail below, A-type lamin proteins include proteins and fragments thereof which have at least 80% sequence identity to a Lamin A, Lamin C or Lamin AΔ10 protein having an amino acid sequence as set out in SEQ ID NO: 1 to 3. The determination of expression of Lamin A/C nucleic acid include the expression of nucleic acid molecules having at least 90% nucleic acid sequence identity to the nucleic acid sequence set out in SEQ ID NO: 4 or which are capable of hybridising to the nucleic acid sequence set out in SEQ ID NO: 4 under stringent conditions.

Preferably, the method of the present invention is an in vitro method carried out on a sample obtained from said individual. In some embodiments of the present invention, the method may therefore comprise an initial step of obtaining a sample from the individual in question and/or preparing the sample for analysis. Preferred examples of samples for use in the method include blood samples, tissue samples or cell samples. The choice of sample may depend on how the determination of A-type lamin status is carried out and can be determined by the skilled person. Examples of suitable tissue samples include a resected tumour sample or tumour biopsy sample. Thus, the method may also include the steps of preparing a sample for analysis from a resected tumour sample or a tumour biopsy sample. The preparation may include the step of staining the sample to enable the A-type lamin status to be determined.

As described herein, the present invention is based on the finding that a reduction in the expression of the Lamin A/C gene, or a loss or a mislocalisation of A-type lamin protein, correlates with a positive prognosis for the individual. In the context, a reduction in the expression of the Lamin A/C gene may mean that there is a lower level of mRNA detectable in the tumour cells, for example as compared to a control of the level of expression in normal cells. Alternatively or additionally, a loss or mislocalisation of A-type lamin protein may mean that the proteins are not observed in their normal localisation pattern in the nuclei of tumour cells. A positive prognosis generally means that the individual is likely to survive in the long term after treatment for the colorectal cancer and/or to have a reduced chance of the recurrence of the colorectal cancer. In other aspects, the present invention is based on the finding that the presence of A-type lamin proteins in a normal nuclear localisation pattern or the expression of Lamin A/C nucleic acid correlates with a negative prognosis, where the negative prognosis may be recurrence of disease or a increased chance of colorectal associated mortality in the individual.

In both instances, the determination of the A-type lamin status in a sample containing colorectal cancer cells may be carried out with reference to one or more controls, although the need for controls will depend on the nature of the test used. Generally speaking, suitable controls can be established by those skilled in the art based on the finding disclosed herein using results previously obtained from one or more other patient samples known to correlate with a particular outcome of colorectal cancer. The skilled person will appreciate that the form of the control that is relevant in a particular test will be dependent on how the A-type lamin status is determined.

By way of example, in the immunohistochemical experiments set out in the examples, a positive control was conveniently provided by the nuclei within the normal interstitial tissue surrounding the tumour. If these nuclei did not stain positively, which occurs in less than 2% of cases, the sample was discarded in favour of a second section. Thus, the positive control in this instance is an internal one. The scoring was then done by determining whether, and what proportion of, the tumour cells nuclei stain positively for A-type lamins. In the examples, a tumour was classified as Lamin A positive, if greater than 10% of the tumour cells nuclei stained positively for A-type lamins.

The A-type lamin status of the individual may be determined in a range of different ways that include, by way of example, (a) determining the presence or amount of an A-type lamin protein, or a fragment thereof, in the sample obtained from the individual, (b) detecting the expression and/or cellular localisation of A-type lamin proteins, (c) detecting or determining the expression of Lamin A/C gene, for example by examining Lamin A/C mRNA levels expressed from the Lamin A/C gene.

Although the invention is exemplified using histological techniques, alternative analysis techniques may be used to determine the expression of the Lamin A/C gene or the presence, absence or localisation of lamin proteins and provide the prognostic information. This may be achieved (i) using immunohistochemical analysis, (ii) by determining the gene and/or protein expression levels of Lamin A/C nucleic acid, (iii) using other forms of protein analysis techniques to detect lamin A/C protein and/or its localisation such as the use of binding assays, protein arrays, Western blotting, mass spectrometry, or other protein detection methods known in the art, (iv) using gene expression technologies such as reverse transcriptase polymerase chain reaction (RT-PCR) to determine mRNA expression levels and/or the presence of Lamin A/C mRNA in a sample and/or (v) by using a microarray technique to determine gene expression.

Preferably, the method comprises detecting the expression and cellular localisation of A-type lamin proteins. Such detection may involve the step of contacting an antibody or antibody fragment capable of recognising said polypeptide, or fragment thereof, with said sample.

The analysis may comprise a qualitative analysis, e.g. by monitoring the presence and cellular localisation of A-type lamin polypeptides by microscopy, e.g. using immunohistochemical staining. Immunohistochemical analysis can be performed on either paraffin fixed samples or on frozen tissue samples.

Examples of possible IHC methods which could be used to detect the lamin A/C polypeptide as described in the present invention.

Preferably, the presence, amount or cellular localisation of A-type lamin proteins may be determined using a binding agent capable of specifically binding to a A-type lamin proteins, or fragments thereof. A preferred type of A-type lamin binding agent is an antibody capable of specifically binding the lamin A proteins or fragment thereof. The antibody may be labelled to enable it to be detected or capable of detection following reaction with one or more further species, for example using a secondary antibody that is labelled or capable of producing a detectable result.

Alternatively, or additionally, the method for determining the A-type lamin status may comprise determining the cellular localisation of an A-type lamin protein, or a fragment thereof, in cells in a sample obtained from the individual. Conveniently, this may be carried out by microscopy and may employ immunohistochemical (IHC) analysis. IHC analysis can be carried out using paraffin fixed samples or frozen tissue samples, and generally involves staining the samples to highlight the presence and location of A-type lamin proteins in the cells.

Alternatively, or additionally, the present invention may involve determining the A-type lamin status by determining the expression of the Lamin A/C gene. In one embodiment, the expression of the Lamin A/C gene can be assessed by determining the presence or amount of lamin A/C mRNA in the sample and methods for doing this are well known to the skilled person. By way of example, they include determining the presence of A-type lamin mRNA in the sample (i) using a labelled probe capable of hybridising to the lamin A/C nucleic acid; and/or (ii) using PCR involving one or more primers based on an A-type lamin nucleic acid sequence to determine whether the A-type lamin transcript is present in a sample. The probe may also be immobilised as a sequence included in a microarray.

In a further aspect, the present invention provides a kit for predicting colorectal cancer associated mortality or recurrence in an individual in accordance with the methods described herein. Preferably, the kit comprises the reagents necessary for carrying out the determination of A-type lamin status on a sample and instructions for carrying out the test and interpreting the results. Preferred types of kit may comprise one or more of the following reagents:
(a) an antibody capable of recognising A-type lamin polypeptides or fragments thereof, for example for use in a binding assay such as an ELISA or in an immunohistochemical test. The antibody may be detected either by being directly labelled or through interaction with one or more other species, for example a labelled secondary antibody; and/or
(b) one or more primers based on the nucleic acid sequence of the lamin A/C gene, for example for detecting the presence of lamin A/C mRNA; and/or
(c) a probe based on the nucleic acid sequence of the lamin A/C gene, for example for detecting lamin A/C gene expression. As for antibody reagents, the probes may conveniently be directly or indirectly labelled to enable them to be detected.

The present invention includes the combination of the aspects and preferred features described except where such a combination is clearly impermissible or is stated to be expressly avoided. Embodiments of the present invention will now be described by way of example and not limitation with reference to the accompanying figures.

### BRIEF DESCRIPTION OF THE SEQUENCES, FIGURES AND TABLES

SEQ ID No: 1-3: Amino acid sequences of the A-type lamin polypeptides encoded by the human Lamin A/C gene.

SEQ ID No: 4: Nucleotide sequence of the human Lamin A/C gene. The start codon is shown in bold type.

FIG. 1: JoL2 antibody staining in normal colon and colorectal cancer (CRC). Tissue sections from normal colon (A) and two different CRCs (B and C) were stained with JoL2 as described in materials and methods. Staining was visualised with DAB using the ABC amplification method. Positive staining was revealed as an intense brown colouration at the nuclear envelope giving a characteristic “halo-like” pattern. In each section I=interstitial tissue. In section A, N=normal colonic epithelium in a transverse section at the top of a crypt. In B and C, T shows a transverse section through a tumour. In C nuclei are visible under phase optics despite the lack of JoL2 staining. NB counter staining with Haemalum was omitted from these sections in order to present a black and white image.

Table 1: Correlation of colorectal cancer mortality with genetic, epigenetic and protein changes in colorectal tumour samples.

Table 2: Association of Lamin A/C expression status with survival in Dukes grade B colorectal cancer patients

Table 3: Association of Lamin A/C expression status with survival in Dukes grade A colorectal cancer patients

### DETAILED DESCRIPTION

Lamin A/C nucleic acid molecules and polypeptides The Lamin A/C gene encodes a number of intermediate type filament proteins, the A-type lamins, which are major components of the nuclear lamina(Hozak 1995). The nuclear lamina is a meshwork of filaments found on the nucleoplasmic side of the nuclear membrane. Nuclear A-type lamins have a range of roles including structural and regulatory functions. The Lamin A/C gene contains 12 exons and alternative splicing within exon 10 of the Lamin A/C messenger RNA gives rise to two proteins; lamin A and lamin C (Lin 1993). The human Lamin A protein is 664 amino acids in length whereas the lamin C protein contains 572 amino acids. The first 566 amino acid residues are common to both lamin A and C. In addition to lamin A and lamin C there is a third splice variant which is identical to lamin A, but which lacks the exon 10 sequence. This form gives rise to a 634 amino acid protein called lamin AΔ10 (Machiels 1996). The amino acid sequences of the human lamin A, lamin C and lamin AΔ10 proteins are shown in SEQ ID NO: 1 to 3 and the nucleotide sequence of the human Lamin A/C gene is shown in SEQ ID NO: 4.

In the present invention, the term “A-type lamin status” includes the determination of the expression of the Lamin A/C gene, and/or the determination of the presence, amount and/or localisation of an A-type lamin protein, or a fragment thereof. A-type lamin proteins include the proteins encoded by the human lamin A/C gene, Lamin A, Lamin C and Lamin AΔ10, the sequences of which are provided herein as SEQ ID NO: 1 to 3. The nucleic acid sequence of the human Lamin A/C gene is provided as SEQ ID NO: 4.

A-type lamin proteins also include polypeptides, proteins or fragments which have an amino acid sequence having a specified degree of sequence identify to SEQ ID No. 1, 2 or 3. Preferred A-type lamin proteins, or fragments thereof, have at least 80% amino acid sequence identity to the Lamin A, Lamin C or Lamin AΔ10 protein having an amino acid sequence as set out in SEQ ID NO: 1 to 3, more preferably at least 90% amino acid sequence identity, more preferably at least 95% amino acid sequence identity, and most preferably at least 98% amino acid sequence identity to a protein having the amino acid sequence set out in any one of SEQ ID NO:1 to 3.

A “Lamin A/C nucleic acid” includes nucleic acid molecules having a nucleotide sequence encoding an A-type lamin protein comprising the amino acid sequence shown in any one of SEQ ID NO: 1 to 3, or any one of the other A-type lamin proteins of the present invention. The Lamin A/C nucleic acid sequence may be the nucleic acid sequence shown in SEQ ID NO: 4 or the portion of SEQ ID NO: 4 that encodes one of the lamin polypeptides, a complementary nucleic acid sequence, or it may be a sequence variant differing from one of the above sequences by one or more of addition, insertion, deletion and substitution of one or more nucleotides of the sequence shown. Changes to a nucleotide sequence may result in an amino acid change at the protein level, or not, as determined by the genetic code. Nucleic acid encoding a polypeptide which is a sequence variant preferably has at least 80% sequence identity, more preferably at least 95% sequence identity, more preferably at least 98% sequence identity, and most preferably at least 99% sequence identity with the nucleic acid sequence shown in SEQ ID NO: 4.

“Percent (%) amino acid sequence identity” with respect to the A-type lamin protein sequences identified herein is defined as the percentage of amino acid residues in a candidate sequence that are identical with the amino acid residues in the A-type lamin sequence, after aligning the sequences and introducing gaps, if necessary, to achieve the maximum percent sequence identity, and not considering any conservative substitutions as part of the sequence identity. The % identity values can be generated by WU-BLAST-2 which was obtained from [Altschul et al, Methods in Enzymology, 266:460-480 (1996); http://blast.wustl/edu/blast/README.html). WU-BLAST-2 uses several search parameters, most of which are set to the default values. The adjustable parameters are set with the following values: overlap span=1, overlap fraction=0.125, word threshold(T)=11. The HSPS and HSPS2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned. region (gaps introduced by WU-Blast-2 to maximize the alignment score are ignored).

Similarly, “percent (%) nucleic acid sequence identity” with respect to the coding sequence of the Lamin A/C nucleic acid sequences is defined as the percentage of nucleotide residues in a candidate sequence that are identical with the nucleotide residues the Lamin A/C coding sequence as provided in SEQ ID NO: 4. The identity values used herein were generated by the BLASTN module of WU BLAST-2 set to the default parameters, with overlap span and overlap fraction set to 1 and 0.125, respectively.

The present invention also includes fragments of the Lamin A/C nucleic acid sequences described herein, the fragments preferably being at least 20, 40, 60, 120, 180, 240, 480 or 960 nucleotides in length. Preferred fragments encode a portion of an A-type lamin protein or may be used as probes for hybridisation to Lamin A/C nucleic acid present in a test sample:

Generally, nucleic acid used in accordance with the present invention may be provided as an isolate, in isolated and/or purified form, or free or substantially free of material with which it is naturally associated. Nucleic acid may be wholly or partially synthetic and may include genomic DNA, cDNA or RNA. Where nucleic acid according to the invention includes RNA, reference to the sequence shown should be construed as reference to the RNA equivalent, with U substituted for T.

The present invention also includes nucleic acid molecules which are capable of hybridising to one of the Lamin A/C nucleic acid sequences disclosed herein, or a complementary sequence thereof, and more particularly are capable of hybridising to SEQ ID NO: 4, or a complementary sequence thereof. Stringency of hybridisation reactions is readily determinable by one of ordinary skill in the art, and generally is an empirical calculation dependent upon probe length, washing temperature and salt concentration. In general, longer probes require higher temperatures for proper annealing, while shorter probes need lower temperatures. Hybridisation generally depends on the ability of denatured DNA to reanneal when complementary strands are present in an environment below their melting temperature. The higher the degree of desired homology between the probe and hybridisable sequence, the higher the relative temperature which can be used. As a result, it follows that higher relative temperatures would tend to make the reaction conditions more stringent, while lower temperatures less so. For additional details and explanation of stringency of hybridisation reactions, see Ausubel et al, Current Protocols in Molecular Biology, Wiley Interscience Publishers, (1995).

Preferably, a nucleic acid sequence will hybridise to a Lamin A/C sequence of the invention, or a complementary sequence thereof under “stringent conditions”. These are well known to those skilled in the art and include those that: (1) employ low ionic strength and high temperature for washing, for example 0.015 M sodium chloride/0.0015 M sodium citrate/0.1% sodium dodecyl sulfate at 50° C.; (2) employ during hybridisation a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 760 mM sodium chloride, 75 mM sodium citrate at 42° C.; or (3) employ 50% formamide, 5×SSC (0.75 M NaCl, 0.075 M sodium citrate), 50 mM sodium phosphate (pH 6 8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

The nucleic acid sequences provided herein are useful for identifying Lamin A/C nucleic acid in a test sample. The present invention provides a method of obtaining nucleic acid of interest, the method including hybridising a probe sharing all or part of the sequence provided herein, or a complementary sequence, to the target nucleic acid. Hybridisation is generally followed by identification of successful hybridisation and isolation of nucleic acid which has hybridised to the probe, which may involve one or more steps of PCR. These methods may be useful in determining whether Lamin A/C nucleic acid is present in a sample, e.g. in a particular type of cells present in the sample.

### Antibodies

The present invention may also employ agents capable of specifically binding A-type lamin polypeptides, for example to help in the determination of the presence, amount or cellular localisation of the A-type lamin polypeptides. A preferred class of binding agents are antibodies. The techniques for producing monoclonal antibodies that are capable of binding A-type lamin polypeptides are a matter of routine in the art. Preferred anti A-type lamin antibodies of the present invention are preferably specific in the sense of being able to distinguish between the one or more of the A-type lamin polypeptides disclosed herein, of fragments thereof, and other human polypeptides for which the antibody has no or substantially no binding affinity (e.g. a binding affinity of about 1000× worse). Specific antibodies bind an epitope on the molecule which is either not present or is not accessible on other molecules. Antibodies used in accordance with the present invention will generally bind to all of the lamin A polypeptides described herein given the fact that their relatively small difference in sequence make it likely that they will contain the same epitope.

Antibodies may be obtained using techniques which are standard in the art. Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and screened, preferably using binding of antibody to antigen of interest. For instance, Western blotting techniques or immunoprecipitation may be used (Armitage et al, Nature, 357:80-82, 1992). Isolation of antibodies and/or antibody-producing cells from an animal may be accompanied by a step of sacrificing the animal. As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the proteins (or fragments), or may be one constructed using sequences obtained from an organism which has been exposed to the antigen of interest.

Antibodies according to the present invention may be modified in a number of ways that are well known in the art. Indeed the term “antibody” should be construed as covering any binding substance having a binding domain with the required specificity. Thus the invention covers antibody fragments, derivatives, functional equivalents and homologues of antibodies, including synthetic molecules and molecules whose shape mimics that of an antibody enabling it to bind an antigen or epitope. Humanised antibodies in which CDRs from a non-human source are grafted onto human framework regions, typically with the alteration of some of the framework amino acid residues, to provide antibodies which are less immunogenic than the parent non-human antibodies, are also included within the present invention.

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP 0 184 187A, GB 2 188 638 A or EP 0 239 400 A. Cloning and expression of chimeric antibodies are described in EP 0 120 694 A and EP 0 125 023 A.

Preferred antibodies for use in accordance with the methods disclosed herein are isolated, in the sense of being free from contaminants such as antibodies able to bind other polypeptides and/or free of serum components. Monoclonal antibodies are preferred for some purposes, though polyclonal antibodies are within the scope of the present invention.

Hybridomas capable of producing antibody with desired binding characteristics are within the scope of the present invention, as are host cells, eukaryotic or prokaryotic, containing nucleic acid encoding antibodies (including antibody fragments) and capable of their expression. The invention also provides methods of production of the antibodies including growing a cell capable of producing the antibody under conditions in which the antibody is produced, and preferably secreted.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule. One favoured mode is by covalent linkage of each antibody with an individual fluorochrome, phosphor or laser exciting dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

### Assays

The determination of A-type lamin status according to the present invention may be carried out in many different ways well known to those skilled in the art that include, by way of example, (a) determining the presence or amount of an A-type lamin protein, or a fragment thereof, in the sample obtained from the individual, (b) detecting the expression and/or cellular localisation of A-type lamin proteins, (c) detecting or determining the expression of Lamin A/C gene, for eXample by examining Lamin A/C mRNA levels expressed from the Lamin A/C gene.

Preferably, the method comprises detecting the expression and cellular localisation of A-type lamin proteins. Such detection may involve the step of contacting an antibody or antibody fragment capable of recognising said polypeptide, or fragment thereof, with said sample.

The analysis may comprise a qualitative analysis, e.g. by monitoring the presence and cellular localisation of A-type lamin polypeptides by microscopy, e.g. using immunohistochemical staining. Immunohistochemical analysis can be performed on either paraffin fixed samples or on frozen tissue samples.

Examples of possible IHC methods which could be used to detect the Lamin A/C polypeptide as described in the present invention.

Alternatively, or additionally, the method for determining the A-type lamin status may comprise determining the cellular localisation of an A-type lamin protein, or a fragment thereof, in cells in a sample obtained from the individual. Conveniently, this may be carried out by microscopy and may employ immunohistochemical (IHC) analysis. IHC analysis can be carried out using paraffin fixed samples or frozen tissue samples, and generally involves staining the samples to highlight the presence and location of A-type lamin proteins in the cells.

Alternatively, or additionally, the present invention may involve determining the A-type lamin status by determining the expression of the Lamin. A/C gene. In one embodiment, the expression of the Lamin A/C gene can be assessed by determining the presence or amount of lamin A/C mRNA in the sample and methods for doing this are well known to the skilled person. By way of example, they include determining the presence of A-type lamin mRNA in the sample (i) using a labelled probe capable of hybridising to the lamin A nucleic acid; and/or (ii) using PCR involving one or more primers based on an A-type lamin nucleic acid sequence to determine whether the A-type lamin transcript is present in a sample. The probe may also be immobilised as a sequence includes in a microarray.

By way of further example a primary antibody that is capable of specifically binding to an A-type lamin protein in a binding assay or an experiment to determine the cellular localisation of the lamin protein may be labelled with a detectable molecule such as, but not limited to, radioactive or fluorescent labels or to enzymes which utilise a chromogenic substrate. Examples of radiolabels of use in this technique are ³²P, ³H or ¹⁴C. Examples of fluorescent molecules of use in this technique are green fluorescent protein, Fluorescein IsoThioCyanate (FITC), Rhodamine IsoThioCyanate (TRICT) Cy3 and Cy5 Dyes. Examples of enzymes with chromagenic substrates of possible use in this technique are peroxidase, alkaline phosphatase or glucose oxidase.

Instead of detecting the signal from the primary antibody itself (as described above), a secondary antibody which binds to the primary antibody can be utilised. The secondary antibody may be labelled with a suitable molecule for detection purposes examples of which are described above.

In an alternative method of detection the primary or secondary antibody may be labelled with a biotin molecule which can then be bound by a streptavidin or avidin linked enzyme with a suitable chromogenic substrate for detection.

Additional variations of the above techniques exist that will be apparent to someone skilled in the art.

One way of detecting the presence of A-type lamin polypeptides and their cellular localisation is by using A-type lamin specific antibodies which may be monoclonal or polyclonal or recombinant antibodies generated, for example, by phage display. These may be used to detect the presence of A-type lamin polypeptides and to visualise their cellular localisation by assays involving binding between A-type lamin polypeptides and the anti-A-type lamin antibody. Suitable antibodies may be labelled with other compounds to aid visualisation, e.g. using fluorescent, chromagenic or radio-labels or secondary antibodies linked to similar labelling compounds.

In the context of this invention antibodies which could be used in such a technique could be generated by standard techniques involving immunisation of animals or could be generated in vitro by recombinant techniques. Antibodies could in this context be whole immunoglobulins or fragments of antibodies (Fab fragments) that correspond to the anti-idiotype. Such antibodies can be readily produced by the skilled person as discussed above.

The invention demonstrates the use of histological analysis to detect Lamin A/C protein and its localisation and from this the prognosis is determined. However, additional methodologies could be employed which could define whether or not Lamin A/C was present in a sample and therefore a poor prognosis was likely. Gene expression technologies such as reverse transcriptase-polymerase chain reaction (RT-PCR) can give accurate measurement of mRNA expression levels and the presence of Lamin A/C mRNA in a sample as opposed to its absence could also be used to provide the prognostic information. RT-PCR can be performed in a range of formats including quantitative versions and with sensitivities that enable the determination of mRNA levels in a single cell. Additional methodologies to detect Lamin A/C gene expression will be apparent to those skilled in the art including a range of gene expression microarray formats that are now widely used. Such technologies make use of probes and primers with homology to the gene of interest which in this case is the Lamin A/C gene as shown in SEQ ID NO: 4.

Although the invention is exemplified using histological techniques, alternative protein analysis techniques could also be used to determine the presence/absence of Lamin A/C and provide the prognostic information. These may include techniques such as protein arrays, western blotting, mass spectrometry as well as other protein detection methods which will be apparent to those skilled in the art.

### Treatment Following Status Determination

Patients falling into a high risk group as defined by their Lamin A/C status would be kept under intensive review for the appearance of tumour markers and monitored by CT scans and regular colonoscopy. These high risk patients could be given adjuvant chemotherapy with agents such as 5-Flurouracil and folonic acid. Although these treatments have some side effects, in the case of Dukes grade B patients this could be justified by the fact that only the high risk grade B patients as identified by their Lamin A/C status would be given the adjuvant chemotherapy.

### Materials and Methods

### Patient Study Population

The patients studied were incident colorectal cancer (CRC) cases and subcohort members from a Dutch prospective cohort study: the Netherlands Cohort Study on diet and cancer (NLCS). The original cohort included 58,279 men and 62,573 women, aged 55-69 years at baseline, all originating from 204 municipal population registries throughout the Netherlands. From 1989 until 1994, 929 incident cases with histologically confirmed Colorectal Cancer (CRC) were identified. For each patient cohort year, within the study period, there was a five year follow-up. Patient material and data, including pathology reports were collected and a CREAM (Colorectal Epidemiology and Mutation) database established to store all corresponding clinical, epidemiological and biological data. A randomly selected cohort of men and women was taken from the CRC cohort and 41 μm sections from paraffin embedded tumour samples were stained using Immunohistochemistry (IHC) for the presence and localisation of Lamin A/C.

### Immunohistochemistry

Patient CRC sections were de-paraffinised in two xylene washes, then re-hydrated in decreasing concentrations of ethanol from 100 to 50%, followed by washing with distilled water. To inhibit endogenous peroxidase activity, tissue sections were treated with freshly prepared 3% H₂O₂ in methanol for 15 min and washed three times with distilled water. Antigen retrieval was carried out by immersing slides into 90° C. 10 mM Citrate buffer (pH 6.0) for 20 minutes. Following antigen retrieval, tissue sections were incubated with a blocking solution (5% normal goat serum in PBS) for 30 minutes at room temperature. Slides were washed with PBS and incubated with the monoclonal primary antibody for Lamin A/C-Jol2 (Dyer 1999) at a dilution of 1:10 overnight at 4° C. Slides were washed three times with PBS, then incubated with a biotinylated goat-antimouse secondary antibody at a concentration of 1:400 for 45 minutes. The Dako ABC Kit (mouse) was used for development of diaminobenzidine tetrahydrochloride (DAB) chromagen to allow detection of the primary antibody. Following incubation with the secondary antibody, the slides were washed three times with PBS and signal amplification using the ABC reagents was carried out according to manufacturer's instructions. The slides were washed with PBS and tissue sections were stained with DAB at a concentration of 0.5 mg/ml in PBS containing 0.01% H₂O₂. Sections were counterstained with Haemalum to highlight nuclei, then dehydrated with increasing concentrations of ethanol from 50 to 100% followed by two washes of xylene and cover-slipped using Entellan histological mounting medium.

### Scoring

Tissue sections were scored blind on two occasions by separate individuals for the presence and localisation of Lamin A/C as shown by DAB staining. Counterstaining with Haemalum was used to show the presence of unstained nuclei. Lamin expression was graded on the presence (N₁) or absence (N₀) of Lamin A/C in the nucleus. The N₁ group included all patient material showing >10% of tumour cell nuclei staining positive for lamin A/C. A note of nuclear staining intensity was also made as well as the presence or absence of cytoplasmic staining. Where IHC had been successful adjacent stromal tissue stained strongly positive and was as such used as an internal control. Sections were then scored blind for tumour architecture based on current pathological staging into 4 groups; Well differentiated, Moderately differentiated, Poorly differentiated and Undifferentiated. The clinicopathological Dukes' stage of the tumours was retrieved from hospital pathology reports via the CREAM database.

### Data Analysis

Once all scoring had been completed, information was retrieved from the CREAM database for patients in question and correlated with scored data. Incidence rate ratios (RR) and corresponding 95% confidence intervals (CI) for CRC-related death with the presence and absence of Lamin A/C were estimated using Cox proportional hazards models. Stratified analysis was also carried out to correlate mortality from CRC related death against Lamin A/C expression across the clinicopathological Dukes' stages. Multivariate analysis of currently held epidemiological, clinical and biological data within the CREAM database against mortality and Lamin A/C expression was also carried out, although in some cases it showed it was not possible to maintain the categorisation due to small numbers of cases per category.

The total person years at risk were also estimated from the sub-cohort. Adjustments for the covariates; age, tumour location, tumour architecture, gender and other biological variables showed no significant effects on findings. The N₁ group was chosen to be the reference group in all analyses. P-values <0.05 were considered to be statistically significant. All analyses were performed with the STATA statistical software package.

### Results

### Lamin Staining in Normal Colon and in CRC

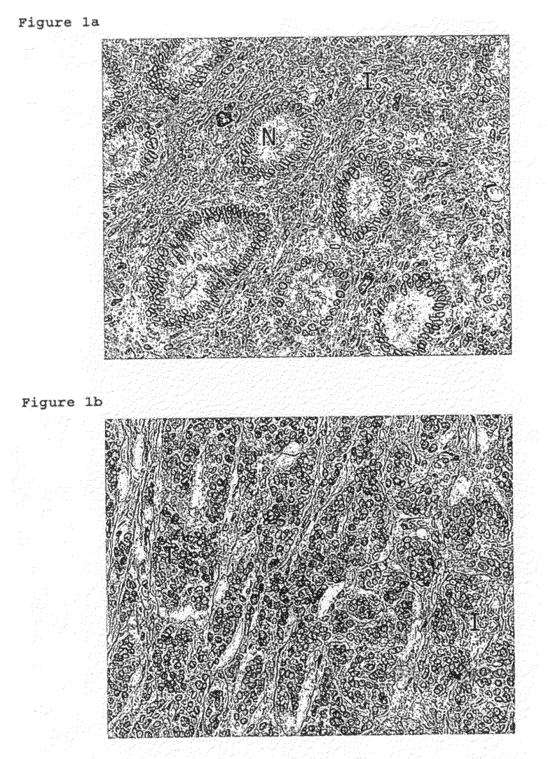

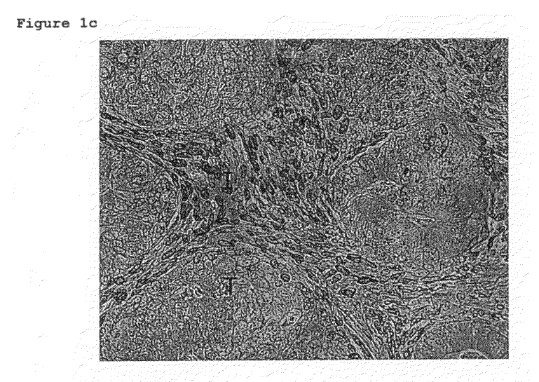

Typical expression patterns for lamin A/C in normal colon are illustrated in FIG. 1A. The anti-lamin A/C monoclonal antibody JoL2 stained the nuclei of differentiated colonic epithelial crypt cells intensely. In addition, the nuclear envelopes within adjacent normal stromal cells were also intensely stained. In tissue from CRC the nuclear envelopes of adjacent normal stromal cells were also always intensely stained and this provided a positive reference within each section (FIGS. 1B and C). Within the cancer two distinct patterns of staining were observed: some cancers had strongly positive staining at the nuclear envelopes of most cells (FIG. 1B). In contrast, other cancers had negative staining at the nuclear envelope, which contrasted starkly with the staining of cells in the surrounding normal stromal tissues (FIG. 1C).

Expression of lamin A/C is correlated with an increased risk of death from colorectal cancer To investigate the association of lamin A/C expression with survival in CRC patients, those patients with high levels of expression of lamin A/C within the tumour who died of CRC related death were compared to those patients with no expression of lamin A/C within the tumour using a Cox proportional harzards model. Patients with Dukes stage A, B or C CRC were combined in order to achieve sufficient numbers for statistical analysis. In this analysis, the group described as No CRC Death, represents all patients who either survived or died from causes other than CRC. The Cox Hazard ratio method assumes that the ratios of individuals that are positive or negative in each group (i.e. No CRC Death and CRC Death) should be equivalent. Therefore, the greater the deviation of the Cox ratio between the two groups, the more highly significant is the data. Table 1 shows that the ratio of lamin A/C +ve/−ve in the No CRC Death group is 257/138 (or 1.86) compared to 97/23 (or 4.22) in the CRC Death group (p=0.001) indicating that the presence of lamin A/C in a CRC is strongly correlated with poor survival.

In order to assess whether lamin A/C expression is a better indicator of prognosis than other markers, Cox Hazard ratios were determined independently for genetic lesions associated with CRC progression. These included methylation of four different promoters, with activating mutations in the Ras proto-oncogene, with inactivating mutations in the APC tumour suppressor gene, micro satellite instability or p53 expression status. There was no significant differences in the Cox Hazard ratios in the No CRC Death compared to the CRC Death groups for any of these markers, suggesting that of those tested, expression of lamin A/C is the only marker with significant prognostic potential (Table 1).

Extracted survival data for Dukes B patients Whilst expression of lamin A/C was indicative of poor prognosis in the entire cohort of Dukes A, B and C patients together, there were too few individuals in the Dukes A and Dukes B sub-groups to statistically analyse independently of Dukes C. Nevertheless sufficient patients were available within the Dukes B sub-group to indicate that the trend in this group was similar or identical to that of the entire cohort. Table 2 shows an extracted data set showing the raw data for the Dukes B sub-group and the trends appear to be very similar.

### Extracted Survival Data for Dukes A Patients

Whilst expression of lamin A/C was indicative of poor prognosis in the entire cohort of Dukes A, B and C patients together, there were too few individuals in the Dukes A and Dukes B sub-groups to statistically analyse independently of Dukes C. Nevertheless sufficient patients were available within the Dukes A sub-group to indicate that the trend in this group was similar or identical to that of the entire cohort. Table 3 shows an extracted data set showing the raw data for the Dukes A sub-group and the trends appear to be very similar.

TABLE 1

| | No CRC | CRC | | p- | Relative |
|---|---|---|---|---|---|
| | Death | Death | Total | value | risk |
| Lack of APC promoter | 224 | 71 | 295 | 0.465 | 0.866 |
| methylation. | | | | | |
| APC promoter methylated. | 150 | 41 | 191 | | |
| Lack of HMLH1 promoter | 291 | 91 | 382 | 0.846 | 0.956 |
| methylation. | | | | | |
| HMLH1 promoter methylated. | 82 | 24 | 106 | | |
| Lack of MGMT promoter | 208 | 69 | 277 | 0.286 | 0.814 |
| methylation. | | | | | |
| MGMT promoter methylated. | 166 | 44 | 210 | | |
| Lack of K-Ras promoter | 55 | 16 | 71 | 0.653 | 1.286 |
| methylation. | | | | | |
| K-Ras promoter methylated. | 10 | 4 | 14 | | |
| Lack of Ras activating | 254 | 70 | 324 | 0.253 | 0.807 |
| mutation. | | | | | |
| Ras activating mutation. | 141 | 48 | 189 | | |
| Lack of APC truncating | 50 | 16 | 66 | 0.767 | 0.923 |
| mutation. | | | | | |
| APC truncating mutation | 345 | 104 | 449 | | |
| Lack of Microsatellite | 357 | 111 | 468 | 0.287 | 0.640 |
| instability. | | | | | |
| Microsatellite Instability. | 36 | 6 | 42 | | |
| Lack of P53 expression. | 179 | 47 | 226 | 0.377 | 1.180 |
| P53 expression. | 215 | 72 | 287 | | |
| Lack of Lamin A/C | 138 | 23 | 161 | 0.001 | 0.465 |
| expression. | | | | | |
| Lamin A/C expression. | 257 | 97 | 354 | | |

Correlation of colorectal cancer mortality with genetic, epigenetic and protein changes in colorectal tumour samples. Ten potential prognostic markers for CRC were investigated including methylation of the APC, HMLH1 and MGMT and K-Ras promoters, activating mutations in the Ras gene, inactivating mutations in the APC gene, microsatellite instability (MSI, expression of p53 and expression of lamins A/C. The p-value is based upon a hazard ratio in which all Dukes grade A, B and C patients are included. Dukes D patients were excluded since >90% died irrespective of the marker.

TABLE 2
Association of lamin A/C expression status
with survival in Dukes B CRC patients.

| | No CRC Death | CRC Death | Total |
|---|---|---|---|
| Lamin A/C +ve | 118 | 35 | 153 |
| Lamin A/C −ve | 43 | 5 | 48 |

CRC related death of Dukes B patients correlates with the presence of (+ve) rather than absence (−ve) of expression of lamin A/C.

TABLE 3
Association of lamin A/C expression status
with survival in Dukes A CRC patients.

| | No CRC Death | CRC Death | Total |
|---|---|---|---|
| Lamin A/C +ve | 75 | 12 | 87 |
| Lamin A/C −ve | 60 | 3 | 63 |

CRC related death of Dukes A patients correlates with the presence of (+ve) rather than absence (−ve) of expression of lamin A/C.

### REFERENCES

The documents disclosed herein are all express incorporated by reference in their entirety.
1. Bulletin of the Royal College of Pathologists 127: July 2004; Sobin L h, Wittekind C H (Eds). TNM Classification of Malignant Tumours (6th Edition) New York: Wiley-Liss, 2002.
2. Bienz, M. and Clevers H. Linking colorectal cancer to Wnt signalling. Cell 2000; 103: 311-320).
3. Gray R G, Barnwell J, Hills C et al. Abstract no 3501. American Society of Clinical Oncology Annual Meeting 2004.
4. Hozak P, Sasseville A M, Raymond Y et al. Lamin proteins form an internal cytoskeleton as well as a peripheral lamina in human cells. Journal of Cell Science 1995; 108: 635-644.
5. Lin F and Worman H J. Structural organization of the human gene encoding nuclear lamin A and nuclear lamin C. Journal of Biological Chemistry 1993; 268: 16321-16326.
6. Machiels B M, Zorenc A H G, Endert J M et al. An alternative splicing product of the Lamin A/C gene lacks exon 10. The Journal of Biological Chemistry 1996; 271: 9249-9253.
7. Broers J L V and Ramaekers F C S. Differential markers for lung-cancer sub-types. A comparative study of expression in vivo and in vitro. International Journal of Cancer. Supplement 1994; 8: 134-137.
8. Machiels B M, Ramaekers F C, Kuijpers H J et al. Nuclear lamin expression in normal testis and testicular germ cell tumours of adolescents and adults. Journal of Pathology 1997; 182: 197-204.
9. Venables R S, McLean S, Luny D et al. Expression of individual lamins in basal cell carcinomas of the skin. British Journal of Cancer 2001; 84: 512-519.
10. Moss S F, Krivosheyev V, de Souza A et al. Decreased and aberrant nuclear lamin expression in gastrointestinal tract neoplasms. Gut 1999; 45: 723-729.
11. Agrelo R, Setien F, Espada J et al. Inactivation of the Lamin A/C gene by CpG island promoter hypermethylation in hematologic malignancies, and its association with poor survival in Nodal Diffuse Large B-cell Lymphoma. Journal of Clinical Oncology 2005; 23: 3940-3947.
12. Dyer J A, Lane E B and Hutchison C J. Investigations of the pathway of incorporation and function of lamin A in the nuclear lamina. Microscopy Research and Technique 1999; 45: 1-12.

1. A method for determining a prognosis of colorectal cancer in an individual, the method comprising determining an A-type lamin status of the individual and using the A-type lamin status to determine the prognosis of the colorectal cancer.

2. The method of any one of the preceding claims, wherein determining the A-type lamin status of the individual comprises determining the expression of the Lamin A/C gene, and/or the presence, amount and/or localisation of an A-type lamin protein, or a fragment thereof.

3. The method of claim 2, wherein the A-type lamin protein, or fragment thereof, has at least 80% sequence identity to a Lamin A, Lamin C or Lamin AΔ10 protein having an amino acid sequence as set out in SEQ ID NO: 1 to 3.

4. The method of claim 2, wherein the Lamin A/C gene has at least 90% nucleic acid sequence identity to the nucleic acid sequence set out in SEQ ID NO: 4 or is capable of hybridising to the nucleic acid sequence set out in SEQ ID NO: 4 under stringent conditions.

5. The method of any one of claims 2 to 4, wherein a reduction in the expression of the Lamin A/C gene in colorectal cancer cells, or a loss or a mislocalisation of A-type lamin protein from the nuclei of colorectal cancer cells, correlates with a positive prognosis.

6. The method of claim 5, wherein the positive prognosis is long term survival and/or a reduced chance of recurrence of the colorectal cancer in the individual.

7. The method of any one of claims 2 to 4, wherein the presence of A-type lamin proteins in a normal nuclear localisation, or normal expression of the Lamin A/C gene, correlates with a negative prognosis.

8. The method of claim 7, wherein the A-type lamin proteins are present in the nuclei of at least 10% of the colorectal cancer cells in a sample.

9. The method of claim 7 or claim 8, wherein the negative prognosis is recurrence of disease or colorectal associated mortality in the individual.

10. The method of any one of the preceding claims, wherein the A-type lamin status is used for predicting the outcome of the colorectal cancer in the individual.

11. The method of claim 10, wherein the outcome is (a) predicting colorectal, cancer associated survival or mortality of the individual and/or (b) predicting the probability of long term survival of the individual and/or (c) predicting recurrence of the colorectal cancer in the individual.

12. The method of any one of the preceding claims, wherein the A-type lamin status is determined with reference to one or more controls.

13. The method of any one of the preceding claims, wherein the A-type lamin status is used for determining clinical treatment of the individual.

14. The method Of claim 13, wherein determining the clinical treatment comprises selecting a type of chemotherapy or a chemotherapy regimen for administration to the individual.

15. The method of claim 14, wherein determining the clinical treatment comprises determining whether a colorectal tumour should be treated by surgical resection.

16. The method of any one of the preceding claims, wherein the A-type lamin status is used for classifying the individual in a grading system for patients with colorectal cancer.

17. The method of claim 16, wherein the grading system is the Dukes scale or the TNM grading system.

18. The method of claim 17, wherein method is for improving the prediction of outcome of patients categorised as grade B on the Dukes scale or the equivalent group of patients in the TNM grading system.

19. The method of claim 17, wherein A-type lamin status is employed for predicting recurrence of early grade or Dukes stage A colorectal cancer or adenomas.

20. The method of any one of the preceding claims, wherein the method is an in vitro method carried out on a sample obtained from said individual.

21. The method of any one of the preceding claims, wherein the method comprises the initial step of obtaining a sample from said individual.

22. The method of claim 20 or claim 21, wherein the sample is a blood sample, a tissue sample or a cell sample.

23. The method of claim 22, wherein the tissue sample is a resected tumour sample or tumour biopsy sample.

24. The method of claim 22 or claim 23, wherein the method comprises the further step of preparing the sample for analysis or staining the cells in the sample for determining the A-type lamin status.

25. The method of any one of the preceding claims, wherein determining the A-type lamin status comprises determining the presence or amount of an A-type lamin protein, or a fragment thereof, in the sample obtained from the individual.

26. The method of claim 25, wherein the method employs a binding agent capable of specifically binding to the A-type lamin protein, or the fragment thereof.

27. The method of claim 25 or claim 26, wherein the binding agent is an antibody capable of specifically binding the A-type lamin polypeptide or fragment thereof.

28. The method of any one of the preceding claims, wherein determining the A-type lamin status comprises determining the cellular localisation of an A-type lamin protein, or a fragment thereof, in cells in a sample obtained from the individual.

29. The method of claim 28, wherein determining the cellular localisation of the A-type lamin protein comprises detecting the presence and cellular localisation of A-type lamin polypeptides by microscopy

30. The method of claim 28 or claim 29, wherein the method employs immunohistochemical analysis.

31. The method of claim 30, wherein the immunohistochemical analysis uses paraffin fixed samples or frozen tissue samples.

32. The method of claim 30 or claim 31, wherein the method comprises staining the samples.

33. The method of any one of the preceding claims, wherein determining the A-type lamin status comprises determining the expression of the lamin A/C gene.

34. The method of claim 33, wherein determining the expression of the lamin A/C gene comprises determining the presence or amount of lamin A/C mRNA in the sample.

35. The method of claim 34, wherein determining the presence of A-type lamin mRNA in the sample comprises:
(i) using a labelled probe capable of hybridising to the lamin A nucleic acid; and/or
(ii) using PCR involving one or more primers based on an A-type lamin'nucleic acid sequence to determine whether the A-type lamin transcript is present in a sample.

36. The method of claim 35, wherein the probe is immobilised in a microarray.

37. A kit for predicting colorectal cancer associated mortality or recurrence in an individual according to the method of any one of the preceding claims.

38. The kit of claim 37, wherein the kit comprises reagents necessary for carrying out the determination of A-type lamin status on a sample and instructions for carrying out the test and interpreting the results.

39. The kit of claim 37 or claim 38, wherein the kit comprises one or more of the following reagents:
(a) an antibody capable of recognising A-type lamin polypeptides or fragments thereof; or
(b) primers based on the nucleic acid sequence of the Lamin A/C gene for detecting the presence of Lamin A/C mRNA; or
(c) a probe based on the nucleic acid sequence of the Lamin A/C gene for detecting Lamin A/C gene expression.


SEQUENCE LISTING (4 sequences)

SEQ ID NO: 1 - protein, 664 aa, Homo Sapiens
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHGSHCSSSGDPAEYNLRSRTVLCGTCGQPADKASASGSGAQVGGPISSGSSASSVTVTRSYRSVGGSGGGSFGDNLVTRSYLLGNSSPRTQSPQNCSIM

SEQ ID NO: 2 - protein, 572 aa, Homo Sapiens
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHVSGSRR

SEQ ID NO: 3 - protein, 634 aa, Homo Sapiens
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEGSHCSSSGDPAEYNLRSRTVLCGTCGQPADKASASGSGAQVGGPISSGSSASSVTVTRSYRSVGGSGGGSFGDNLVTRSYLLGNSSPRTQSPQNCSIM

SEQ ID NO: 4 - DNA, 3181 bp, Homo Sapiens
actcagtgttcgcgggagcgccgcacctacaccagccaacccagatcccgaggtccgacagcgcccggcccagatccccacgcctgccaggagcaagccgagagccagccggccggcgcactccgactccgagcagtctctgtccttcgacccgagccccgcgccctttccgggacccctgccccgcgggcagcgctgccaacctgccggccatggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaagcttgaggcagccctaggtgaggccaagaagcaacttcaggatgagatgctgcggcgggtggatgctgagaacaggctgcagaccatgaaggaggaactggacttccagaagaacatctacagtgaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaagctggacaatgccaggcagtctgctgagaggaacagcaacctggtgggggctgcccacgaggagctgcagcagtcgcgcatccgcatcgacagcctctctgcccagctcagccagctccagaagcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggaccagtccatgggcaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacggctcccactgcagcagctcgggggaccccgctgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccagagcccccagaactgcagcatcatgtaatctgggacctgccaggcaggggtgggggtggaggcttcctgcgtcctcctcacctcatgcccaccccctgccctgcacgtcatgggagggggcttgaagccaaagaaaaataaccctttggtttttttcttctgtatttttttttctaagagaagttattttctacagtggttttatactgaaggaaaaacacaagcaaaaaaaaaaaaaagcatctatctcatctatctcaatcctaatttctcctcccttccttttccctgcttccaggaaactccacatctgccttaaaaccaaagagggcttcctctagaagccaagggaaaggggtgcttttatagaggctagcttctgcttttctgccctggctgctgcccccaccccggggaccctgtgacatggtgcctgagaggcaggcatagaggcttctccgccagcctcctctggacggcaggctcactgccaggccagcctccgagagggagagagagagagagaggacagcttgagccgggcccctgggcttggcctgctgtgattccactacacctggctgaggttcctctgcctgccccgcccccagtccccacccctgcccccagccccggggtgagtccattctcccaggtaccagctgcgcttgcttttctgtattttatttagacaagagatgggaatgaggtgggaggtggaagaagggagaagaaaggtgagtttgagctgccttccctagctttagaccctgggtgggctctgtgcagtcactggaggttgaagccaagtggggtgctgggaggagggagagggaggtcactggaaaggggagagcctgctggcacccaccgtggaggaggaaggcaagagggggtggaggggtgtggcagtggttttggcaaacgctaaagagcccttgcctccccatttcccatctgcaccccttctctcctccccaaatcaatacactagttgtttct